(19) United States
(12) Patent Application Publication
(10) Pub. No.: US 2012/0020932 A1
(43) Pub. Date: Jan. 26, 2012

(54) THERMOSENSITIVE HYDROGEL COMPOSITION AND METHOD

(75) Inventors: Jian Q. Yao, Shanghai (CN); Jizong Gao, Cedar Park, TX (US); Xiao Huang, Freehold, NJ (US); Archit Sanghvi, North Richland Hills, TX (US)

(73) Assignee: Zimmer Orthobiologics, Inc., Austin, TX (US)

(21) Appl. No.: 13/174,107

(22) Filed: Jun. 30, 2011

Related U.S. Application Data
(60) Provisional application No. 61/361,164, filed on Jul. 2, 2010.

(51) Int. Cl. A61K 35/12; A61K 38/17; A61P 43/00; A61K 38/39

(52) U.S. Cl. 424/93.7; 514/17.2; 514/1.1

(57) ABSTRACT

A hydrogel-forming composition is provided that comprises an extracellular matrix protein, hyaluronic acid, and a thermosensitive biocompatible polymer such as methylcellulose. The hydrogels can provide a therapeutic effect; further, the hydrogels may comprise an optional therapeutic agent such as cells or a pharmaceutical composition. The composition may be injected to an area in need of treatment by the therapeutic agent. The composition may form a gel at about 37° C., such that the gel maintains the therapeutic agent in the area of the body in need of such treatment.

### CROSS-REFERENCE TO RELATED APPLICATIONS

This application claims the benefit of U.S. Provisional Application No. 61/361,164, filed on Jul. 2, 2010, under 35 U.S.C. §119(e), which is incorporated herein by reference in its entirety.

### BACKGROUND OF THE INVENTION

The term “hydrogel” refers to a broad class of polymeric materials which are swollen extensively in water, but which do not dissolve in water. Generally, hydrogels are formed by polymerizing a hydrophilic monomer in an aqueous solution under conditions wherein the polymer becomes cross-linked so that a three-dimensional polymer network is formed which is sufficient to gel the solution. Hydrogels have many desirable properties for biomedical applications. For example, they can be made nontoxic and compatible with tissue. In addition, they are usually highly permeable to water, ions and small molecules.

In the healing of localized injury or disease, localized delivery of therapeutic agents can be advantageous compared to systemic delivery of therapeutic agents, because systemic delivery can require much higher doses of the therapeutic agent and can result in undesirable side effects. Thus medical research has been directed to means and methods for localized delivery of therapeutic agents. Such means and methods include bolus injection, minipump delivery, and injectable gels.

Injectable gels can be either physical gels or chemical gels. Chemical gels require in situ cross linking which can involve cytotoxic cross-linkers or free radicals. Physical gels are compositions which form gels due to the change of some physical characteristic such as temperature. Examples of physical gels include methyl cellulose and hyaluronic acid. Hyaluronic acid is a naturally occurring polymer of disaccharides linked by glycosidic bonds. It is found in both cartilage and in skin, where it is present in extracellular matrices. It is naturally involved in tissue repair. Because of its biocompatibility and natural presence in the extracellular matrix of tissues, it is known as a biomaterial scaffold in tissue engineering research, such as in artificial skin. Methyl cellulose is a hydrophilic group of synthetic polymers derived from the polymer cellulose. Methyl cellulose solutions have inverse thermal gelling properties, being a liquid when cool and forming a gel as temperature increases. Methyl cellulose has shown good biocompatibility when used as scaffolds in traumatic brain injury and peripheral nerve regeneration. Gupta et al., Biomaterials vol. 27, no. 11, pp. 2370-2379 (2006) states that methyl cellulose alone is not sufficiently fast gelling to be useful as an injectable drug delivery system for spinal cord injuries. Gupta et al. discloses injectable blends of hyaluronic acid and methyl cellulose for intrathecal delivery, and states that the compositions gel faster than methyl cellulose alone.

### SUMMARY OF THE INVENTION

This invention relates to injectable compositions to promote healing and/or tissue regeneration. More particularly, this invention relates to injectable compositions that optionally contain therapeutic agents, which compositions are liquid at ambient temperature and form hydrogels at internal body temperatures to provide therapy to areas of the body in need of tissue repair and/or regeneration. The hydrogels of the invention gradually degrade in vivo through polymer dissolution and/or gradual non-specific enzyme degradation.

The invention thus provides a biocompatible composition for providing a scaffold for tissue regeneration, and a biocompatible composition and method of delivering therapeutic agents to different parts of the body. For example, joint structures can undergo injury or damage such as osteoarthritis in the cartilage and meniscal tissues to provide a biocompatible structure that promotes healing and/or tissue regeneration. The hydrogels of the invention provide for local delivery of therapeutic agents such as pharmaceutical agents, growth factors, or cells, directly to a site requiring treatment, e.g., to create an optimal regeneration environment.

In one embodiment, the invention provides a hydrogel-forming composition comprising a biocompatible non-protein thermosensitive polymer, isolated hyaluronic acid, an isolated extracellular matrix protein, and optionally an aqueous medium. The composition is substantially liquid at ambient temperature and gelable at temperatures above ambient temperature, e.g., at 37° C. The extracellular matrix protein can be selected from any one or more of type I collagen, type II collagen, type A gelatin, type B gelatin, and other suitable protein materials. The non-protein portion of the composition is thermosensitive. In one embodiment, the thermosensitive polymer comprises methyl cellulose. Other representative non-protein, thermo sensitive polymers include: poly-N-isopropylacrylamide (NiPAAM); poly(vinyl alcohol); poly(NiPAAM)/poly(ethylene glycol); poly(ethylene oxide-propylene oxide-ethylene oxide) (PEO-PPO-PEO); and poly(ethylene glycol-lactic acid-ethylene glycol) (PEG-PLLA-PEG). The aqueous medium can be water or an aqueous solution such as a phosphate buffered saline solution.

One advantage of the present invention is that the composition is a flowable liquid at ambient temperature yet forms a soft gel at temperatures of about 37° C. A therapeutic agent such as a pharmaceutical composition or cells optionally can be combined with the composition.

In one embodiment, the invention provides an injectable hydrogel-forming composition that can form a hydrogel in situ upon injection. In one embodiment, the invention provides an injectable hydrogel-forming composition that can be injected as a hydrogel. In one embodiment, the invention provides an injectable hydrogel-forming composition that either can form a hydrogel in situ upon injection, or can be injected as a hydrogel, which composition is capable of delivering therapeutic agents to an injection site. In one embodiment, the invention provides an injectable hydrogel-forming composition that can form a hydrogel in situ upon injection, or can be injected as a hydrogel, and which is biodegradable by hydrolysis and/or enzymatic degradation. In one embodiment, the invention provides an injectable hydrogel composition that either forms a hydrogel in situ or can be injected as a hydrogel, and which includes both a non-protein polymer and an isolated extracellular matrix protein. In one embodiment, the invention provides a method of preparing a hydrogel-forming composition.

In accordance with one method of making the composition of the invention, a hydrogel-forming composition suitable for injection, e.g., for injection in sites other than the intrathecal region, is prepared by providing a solution of hyaluronic acid in an aqueous medium, adding a quantity of biocompatible thermosensitive polymer with mixing, adding an extracellular matrix protein to the aqueous mixture, then adding additional aqueous medium with continued mixing at a temperature beneath the gel-forming temperature. The aqueous medium may be a phosphate buffered saline solution.

In one embodiment, the invention provides a method of using a hydrogel-forming composition to provide therapy to a site in need thereof, including providing optional therapeutic agents to a site in need of such agents.

The hydrogel of the present invention may serve as a scaffold, e.g., for tissue regeneration, or a void-filler. It may also supplement the viscous properties of the synovial fluid within an affected joint. Optionally, a therapeutic agent, such as pharmaceutical agents, growth factors, or cells, can be added to the composition to promote healing. When the optional therapeutic agent to be delivered by the injectable hydrogel is cells, the hydrogel may improve cell adhesion, improve cell proliferation, and/or improve cell differentiation in the hydrogel environment. A hydrogel-forming composition for the delivery of therapeutic agents, including cells, may be both biocompatible and biomimetic, thereby providing biological clues to the therapeutic cells being delivered and to the surrounding tissue. Moreover, the local delivery of therapeutic agents such as pharmaceutical agents, growth factors, or cells directly to a site requiring treatment may create an optimal regeneration environment.

In accordance with a method of using the hydrogen-forming composition, in one embodiment, the composition is maintained at a temperature below 37° C. and then injected, whereupon it forms a hydrogel in situ at the warmer temperature of the injection site. In another embodiment of a method of using the composition, the composition is first warmed to a temperature to form a hydrogel, and then injected as a gel to the area in need of treatment. In accordance with either method of use, any optional therapeutic agent present in the composition is maintained in contact with the affected area to promote healing thereof.

### DESCRIPTION OF THE FIGURES

FIG. 1 is graph showing the mass change of a hydrogel of the present invention as compared to two prior art hydrogel compositions over a 14 day period.

FIG. 2 is a standard curve using DNA from a PicoGreen kit and shows the results of DNA measurements using the PicoGreen kit on samples of hydrogels containing equine chondrocytes, measured at intervals of 1, 3, 7, 14, and 21 days.

FIG. 3 is a graph showing complex modulus data for hydrogel formulations of the present invention, with and without added chondrocytes.

### DETAILED DESCRIPTION OF THE INVENTION

A hydrogel-forming composition suitable for administration by injection to a localized site includes a biocompatible non-protein thermosensitive polymer, isolated hyaluronic acid, and an isolated extracellular matrix protein. An “isolated” biomolecule includes one that is prepared in vitro, e.g., a recombinant biomolecule, or is separated from at least one contaminant with which it is ordinarily associated within its source, e.g., present in a form or setting that is different from that in which it is found in nature. When combined with an aqueous medium, the composition forms a hydrogel when the mixture is brought to an appropriate temperature. The composition with aqueous medium remains a liquid at ambient temperatures. Upon injection to an area in need of treatment, the mixture flows about the area, and, in the warmer environment of the injection site, forms a stable hydrogel. Alternatively, the mixture can be warmed to form a hydrogel prior to injection.

The hydrogel can serve as a scaffold or void filler to promote regeneration of tissue. It also can supplement the viscous properties of the synovial fluid within an affected joint. In one embodiment, the hydrogel-forming composition further comprises a therapeutic agent, which can be cells, a pharmaceutical composition, or both. The stable hydrogel maintains the therapeutic agent in the composition in proximal relation to the area in need of such therapy.

In a hydrogel-forming composition of the invention, the biocompatible non-protein thermosensitive polymer may be methyl cellulose. Other representative non-protein, thermosensitive polymers include: poly-N-isopropylacrylamide (NiPAAM); poly(vinyl alcohol); poly(NiPAAM)/poly(ethylene glycol); poly(ethylene oxidepropylene oxide-ethylene oxide) (PEO-PPO-PEO); and poly(ethylene glycol-lactic acid-ethylene glycol) (PEG-PLLA-PEG). Other biocompatible non-protein thermosensitive polymers will be known to those skilled in the art of hydrogels. In particular, the thermosensitive polymer component will be one which helps to maintain the composition in the liquid state at ambient temperatures, and promotes formation of a gel at temperatures at or near that of a mammal, e.g., the temperature of the human body. Two or more non-protein thermosensitive polymers can be used in a single hydrogel composition The gel formation is the result of polymerization of the thermosensitive component in an aqueous medium, e.g., a buffered saline solution, at temperatures of about 37° C., the temperature of the human body.

The extracellular matrix protein may be structural proteins such as collagen Type I, collagen Type II, and elastin; specialized proteins such as fibrillin, fibronectin, and laminin; proteoglycans, e.g., combinations of carbohydrates and proteins; and gelatins, including Type A gelatins, type B gelatins, and mixtures thereof, and native gelatins from sources such as bovine skin, porcine skin, and fish skin. The extracellular matrix also can comprise combinations of any two or more of the foregoing types of proteins.

The hydrogel-forming composition is based on an aqueous medium. In a one embodiment, the aqueous medium is a phosphate buffered saline (PBS) solution, e.g., one having about 0.010 M PBS.

In some embodiments of the invention, as a percentage of solids (exclusive of optional therapeutic agent), the hydrogel-forming composition of the present invention can include about 70% to about 95% by weight of biocompatible non-protein thermosensitive polymer, about 4% to about 30% by weight of hyaluronic acid, and about 0.01% to about 3% by weight of extracellular matrix protein. In some embodiments of the invention, the weight ratio of thermosensitive polymer to hyaluronic acid can be in the range of about 2:1 to about 10:1, including any integer between 2 and 10 for the first numerical value in the ratio.

When determined as weight % per volume upon addition of an aqueous medium (exclusive of optional therapeutic agent) in some embodiments of the invention, the hydrogel-forming composition of the present invention can include about 2 w/v % to about 12 w/v % of biocompatible non-protein thermosensitive polymer, about 0.2 w/v % to about 6 w/v % of hyaluronic acid, and about 0.001 w/v % to about 0.5 w/v % of extracellular matrix protein.

It will be understood by those skilled in the art that the ranges and ratios stated above are approximate, and may depend on the choices of thermosensitive polymer and extracellular matrix protein, as well as on the rheological, gelation and degradation properties desired in the hydrogel product, and the presence or absence of optional therapeutic agents.

In some embodiments, the hydrogel-forming composition can further comprise one or more biocompatible components to achieve certain desired mechanical properties, such as modulus and viscosity. Polyethylene glycol may be suitable for this purpose; other suitable polymers will be known to those skilled in the art of biocompatible hydrogels. The proportions of the components of the hydrogel-forming composition can be adjusted to achieve a hydrogel of desired mechanical properties, such as modulus and viscosity. For example, rheological studies suggest that polyethylene glycol (PEG) (e.g., PEG at about 7.5 kDa) enhances the mechanical strength of 8% methyl cellulose (MC)-based hydrogel without dramatically increasing the viscosity of the precursor solution. The effect of adding various amounts of PEG, from 0% to about 10%, into about 8% MC can dramatically increase the storage modulus of the hydrogel when the temperature is above about 30° C., but the effect on the loss modulus is minor as compared to the storage modulus. It is believed that such modifiers may have the same effect on the methylcellulose/hyaluronic acid/extracellular matrix protein-containing hydrogel compositions of the present invention. Other suitable modifiers may include chondroitin sulfate, chitosan, dextrans, other polysachharrides, and other natural and synthetic polymers. Other modifiers will be recognized by those of skill in the art. Those skilled in the art also will be able to determine the optimum range of molecular weights of such modifier polymers by routine experimentation for each hydrogel-forming composition of the present invention.

When the optional therapeutic agent to be delivered by the injectable hydrogel includes cells, the extracellular matrix protein present in the hydrogel-forming composition may function to improve cell adhesion, improve cell proliferation and/or improve cell differentiation in the hydrogel environment. Cytocompatibility may be determined by measuring the viability of cells by known techniques and commercially available products such as by staining with a Live/Dead® Viability/Cytotoxicity Kit (Invitrogen) (calcein AM/ethidium homodimer). Cell proliferation was measured by quantifying DNA content. Increasing natural components of the hydrogel, such as gelatin or collagen, may yield improved cell adhesion. Without wishing to be bound by theory, it is believed that the extracellular matrix protein is both biocompatible and biomimetic, enhancing cell activity by providing an optimal cell environment that mimics the natural extracellular matrix protein of connective tissue, thereby providing biological cues to the therapeutic cells being delivered and to the surrounding tissue; it is further believed that such cues can include signals for cell-matrix interactions, or growth factor receptors. Specific growth factors in a particular medium may induce specific cellular phenotypes.

In one method of use, the hydrogel-forming composition is maintained at a temperature of less than about 37° C. to keep it in a liquid state, and injected into the body as a liquid at a site requiring such therapy, where it forms a hydrogel when at the temperature of the human body, about 37° C.

In an alternative method of use, the hydrogel-forming composition is brought to a temperature of about 37° C. to form a gel, and then injected as a gel to the site requiring therapy. The composition may be loaded into a syringe as a liquid, warmed in the syringe to form a gel, and injected from the syringe as a gel to the affected site. Alternatively the composition may be in the gel state when it is loaded into the syringe.

In accordance with one embodiment of a method of making the composition of the invention, a hydrogel composition suitable for injection is prepared by preparing a solution of hyaluronic acid in an aqueous medium, adding a quantity of biocompatible thermosensitive polymer with mixing, adding an extracellular matrix protein to the aqueous mixture, then adding additional aqueous medium with continued mixing at a temperature beneath the gel-forming temperature. In one embodiment, the aqueous medium is a phosphate buffered saline (PBS) solution. A therapeutic agent may be added to the composition. In another embodiment of the method, a portion of the biocompatible thermosensitive polymer can be added before the hyaluronic acid is added. In one embodiment, the extracellular matrix protein may be added in the form of a solution. In one embodiment of the method of making the hydrogel-forming compositions of the present invention, the thermosensitive polymer is maintained below its thermoset temperature until it is completely solubilized. In one embodiment of the inventive method of preparing the composition, a quantity of hyaluronic acid is added to a quantity of 0.010 M PBS in a vessel at ambient temperature and stirred until the acid is dissolved. The solution is then heated to about 90° C., and a desired quantity of methylcellulose is added to the vessel. In one embodiment, the methylcellulose is added to a portion of the solvent at an elevated temperature with mixing to ensure that the methylcellulose aggregates are thoroughly wetted and dispersed. The remaining solvent is then added at a lower temperature to promote solubility of the powder in the aqueous solvent. The vessel is then cooled, and an amount of extracellular matrix protein is added. The extracellular matrix protein can be added as a solid or as a solution. The contents are mixed, and the vessel is cooled. Cold phosphate buffered saline is then added with mixing to bring the composition to the desired concentration level.

The composition of the present invention advantageously forms a biocompatible hydrogel without the introduction of chemical cross-linkers or free radicals. The injection procedure also is minimally invasive, resulting in the least possible disruption of host tissues and structures. The composition of the present invention may be safely injected into many areas of the body, including, for example, diarthrodial joints, intervertebral disks, facet joints, and skin.

The invention will be further described by the following non-limiting examples.

### Example 1

In all the Examples herein, rheological properties including storage modulus (G′), loss modulus (G″), and complex modulus (G*) were measured on a model MCR 101 rheometer available from Anton Paar using a 25 mm serrated plate. The hydrogel samples were in the form of cylinders having a thickness of about 1 to about 2 mm and a diameter slightly greater than the rheometer plate. The top and bottom surfaces of the cylinder sample were flat. The bottom plate temperature was about 35 to about 37° C. The sample was placed between the bottom plate and the serrated plate with 600 grit sandpaper.

The amplitude sweep was used to test the linear range of the hydrogel, and the frequency sweep was used to test the storage, loss, and complex modulus. The strain was at about 1% if the hydrogel had a linear range over about 1%; otherwise, a lower strain was used. The frequency range was about 0.1 to about 100 s-1. Twenty-four measuring points were taken for each sample. There was an interval of at least five minutes between each test.

### Preparation of Methyl Cellulose-Hyaluronic Acid-Type I Collagen Hydrogel Forming 20 Composition

In a beaker, 150 mL of sterile 1× Dulbecco's phosphate-buffered saline (D-PBS) is heated to 90° C. To the heated saline is added 8 g methyl cellulose powder (Sigma, catalog #M7140), then 4 g hyaluronic acid (Engelhard), then another 4 g of methylcellulose powder, with through mixing after each addition, about 30 minutes of mixing. To this heated mixture is added about 4.5 mL of a stock solution of about 7 mg/mL type I collagen (BD Biosciences, catalog #354249, lot #15426), with continued mixing for 10 minutes. Immediately, 50 mL of 1×PBS at 4° C. is added to obtain a volume of 200 mL. Mixing continued at 4° C. in an ice bath for 30 minutes. The final mixture is stored overnight at 4° C. prior to use. Gels are formed by aliquotting about 3 to about 4 mL samples of the 4° C. solutions into 24-well plates and incubating the plates at 37° C. for 24 hours.

Certain rheological properties of the composition of Example 1 are compared with a first control hydrogel composition comprising 8% methyl cellulose and a second control hydrogel composition comprising 8% methylcellulose and 4% hyaluronic acid; neither of the control compositions included a matrix protein. The results are summarized in Table I.

TABLE 1
Gelation time, temperature, and complex modulus

| | | 8% | |
|---|---|---|---|
| | 8% | methylcellulose- | |
| | methylcellulose | 4% hyaluronic acid | |
| | (control) | (control) | Example 1 |
| Gelation time | about 70 | about 30 | about 20 |
| @35° C. | | | |
| (seconds) | | | |
| Gelation | 32 | 28 | 26 |
| temperature | | | |
| (° C.) | | | |
| Complex | 800 ± 18 | 1247 ± 84 | 1308 ± 100 |
| modulus @ 1% | | | |
| strain, 10 Hz | | | |
| (Pa) | | | |

Gelation time is determined at the point at which the storage modulus is greater than the loss modulus. Gelation temperature is measured by increasing the temperature of a sample of the hydrogel from about 4° C. to about 40° C. at a rate of about 2° C./minute. The results indicate that the composition of the present invention forms a hydrogel faster and at a lower temperature than other hydrogel-forming compositions. Further, the hydrogel formed from the hydrogel-forming composition of the invention is stronger than the gel formed using only methylcellulose, as measured by the complex modulus.

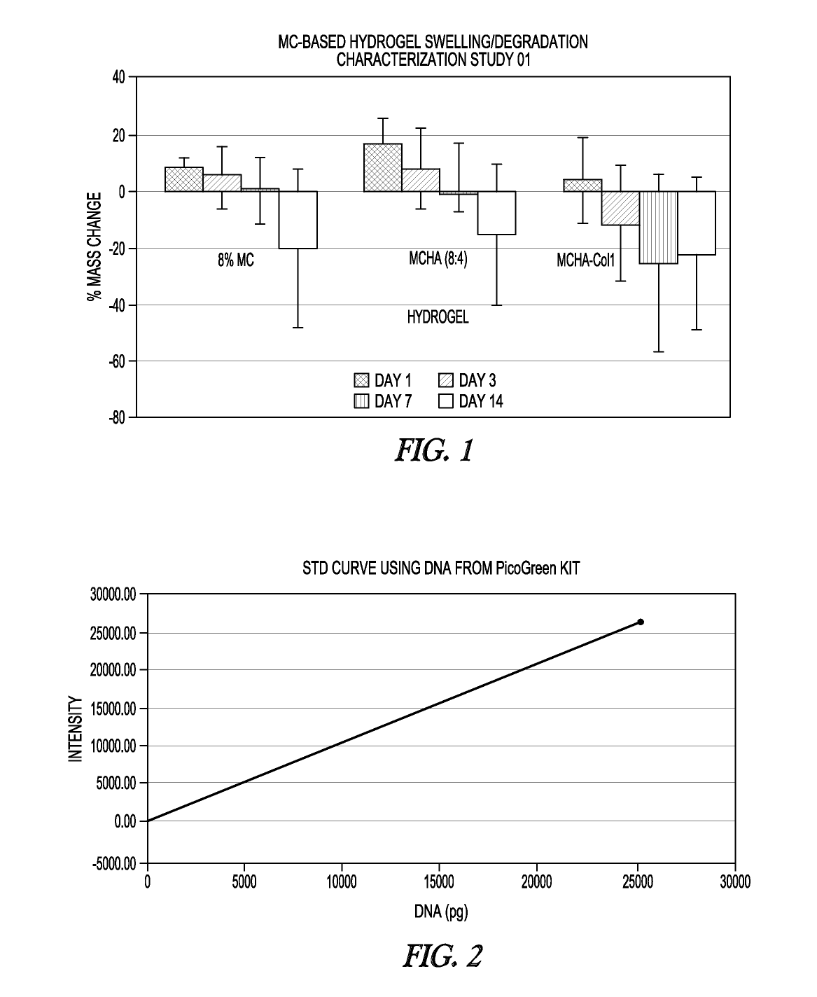

The swelling and degradation of the three hydrogels is measured. Samples of each hydrogel are prepared by aliquotting 3 mL volumes at 4° C. into 12-well plates, and then incubating at 37° C. Each sample is removed from the well plate, weighed, and placed in 2-3 ml DMEM/F12 tissue culture medium supplemented with 10% fetal bovine serum (both from Gibco BRL). The samples are weighed at pre-determined intervals to determine the amount of swelling or degradation that occurs. The results are illustrated in FIG. 1. It may be seen that each of the gels shows some swelling in the first day, while after seven days the two control gels will return to about their original mass and the hydrogel of the present invention starts to degrade, as indicated by the reduction in mass.

### Example 2

### Preparation of Methyl Cellulose-Hyaluronic Acid-Type II Collagen Hydrogel Forming Composition

In a beaker, 150 mL of sterile 1× Dulbecco's phosphate-buffered saline (D-PBS) is heated to 90° C. To the heated saline are added 8 g methyl cellulose powder (Sigma, catalog #M7140, 14,000 m.w.), then 2 g hyaluronic acid (Engelhard, catalog #HA-501 100-1, batch #565608), then another 8 g of methylcellulose powder, with thorough mixing after each addition, for about 30 minutes of mixing. To this heated mixture is added about 5 mg of type II collagen as a stock solution of about 2.98 mg/mL (BD Biosciences, catalog #354257, lot #008794), with continued mixing for 10 minutes. Immediately, 50 mL of 1×PBS at 4° C. is added to obtain a volume of 200 mL. The mixture contains 8% methylcellulose, 2% hyaluronic acid, and 0.0025% collagen type II, measured as w/v. Mixing continues at 4° C. in an ice bath for 30 minutes. The final mixture is stored overnight at 4° C. prior to use.

The hydrogels of Example 1 and Example 2 are found to have greater swelling over periods of 1 day, 2 days, 3 days, and 7 days compared to a 8% methyl cellulose gel and a 8% methylcellulose/1% hyaluronic acid gel.

Hydrogels of the present invention exhibit favorable gelation times and favorable modulus properties relative to hydrogels of only methylcellulose or hydrogels of methylcellulose with hyaluronic acid, as set forth in Table 2 below. In the data reported therein, the gelation onset time is measured with gels starting at 4° C. placed on the rheometer with the bottom plate at 35° C., and the top plate adjusted to a height of one mm. Measurements are performed with strain at 1% and frequency at 10 Hz. The gelation onset time is determined as the intersection between the loss and storage modulus. The modulus at complete gelation is determined by analyzing the complex modulus curve of each hydrogel; the point at which a plateau is reached is determined to be the post-gelation modulus.

TABLE 2
gelation time, modulus

| | Gelation | | | Complex |
|---|---|---|---|---|
| | onset | Time to | | modulus |
| | time | complete | Modulus at | post- |
| | at 35° C. | gelation at | complete | gelation |
| | (min) | 35° C. (min) | gelation (Pa) | 35° C. (Pa) |
| 8% MC | 7 | 48 | 921 | 1700 |
| MC:HA (8:1) | 4.75 | 30 | 1238 | 2280 |
| MC:Gelatin (8:1) | 4.25 | 32 | 1165 | 2120 |
| MC:HA:Col I | 3.25 | 22 | 1018 | 1300 |
| (8:1:0.1) | | | | |
| MC:HA:Col. II | 1.5 | 31 | 5911 | 7400 |
| (8:1:0.25) | | | | |

### Example 3

### Preparation of Methyl Cellulose-Hyaluronic Acid-Type B Gelatin Hydrogel Forming Composition

All equipment and materials are sterilized in an autoclave by known methods to insure a sterile composition. In a 1 L media bottle with a mechanical stirrer is placed 300 mL of 1×PBS. To the bottle are added 2 g hyaluronic acid powder; the bottle is capped and the contents of the bottle are stirred continuously for 48 to 72 hours to ensure complete dissolution. The solution is then heated in a hot water bath to 90° C., with stirring. To the heated solution, 32 g of methylcellulose was slowly added; the contents were mixed while the bottle was loosely capped. This is followed by immediate vigorous shaking with the bottle tightly capped; the cap is then released briefly to release pressure. The bottle is place on ice to cool for about ten minutes. To the bottle is added type B gelatin (Sigma Aldrich) in the amount of 400 mg for a 0.1% w/w end product. To the bottle is then added 100 mL of 0.010 PBS at 4° C. to bring the contents to the final desired concentration, the bottle is shaken vigorously to ensure proper mixing. The bottle is immediately covered with ice and incubated for or twenty minutes, with periodic shaking. The solution is stored at 4° C.

### Example 4

### Preparation of Methyl Cellulose-Hyaluronic Acid-Protein Hydrogel Forming Compositions

Hydrogels are prepared using methyl cellulose, hyaluronic acid and a protein selected from Type A gelatin, Collagen I, and Collagen II. All equipment and materials are sterilized in an autoclave by known methods to insure a sterile composition. The following materials are used:

Methylcellulose, Sigma catalog M1740, autoclave sterilized;
Hyaluronic acid, Engelhard catalog #R10271, MW about 1.5 to about 2.0 million Da;
Rat tail collagen 1, BD Biosciences catalog #354249, high cone. (about 8 mg/ml);
Bovine collagen II, BD Biosciences catalog #354257, 5 mg/mL vials;
Gelatin Type A porcine, Sigma approx. 300 bloom;

### 1×PBS, Gibco.

The following procedure is used for all the gels:
In a 1 L media bottle with a mechanical stirrer is placed 300 mL of 1×PBS. To the bottle, 2 g hyaluronic acid powder are added; the bottle is capped and the contents of the bottle are stirred continuously for 48 to 72 hours to ensure complete dissolution. The solution is then heated in a hot water bath to 90° C., with stirring. To the heated solution, 16 g of methylcellulose are slowly added; the contents are mixed while the bottle is loosely capped. Where the protein is gelatin, then 400 mg of autoclave-sterilized gelatin is added at this time. The remaining 16 g of sterile methylcellulose is then added with stirring for about 5 to 10 minutes or until completely mixed without the presence of any clumps. The bottle is vigorously shaken, if necessary, to dislodge clumps. The remaining 100 mL of 10 mM PBS chilled to 4° C. is added with vigorous shaking to ensure proper mixing. Where the protein is either Collagen I or Collagen II, then either 100 mg of Collagen 1 or 10 mg Collagen II is added at this time, with vigorous shaking to ensure complete mixing. The resulting mixtures are stored overnight at 4° C. A control is also prepared with methylcellulose and hyaluronic acid and no protein component. The compositions of the four formulations prepared are set forth in Table 3. Formulation 1 contains no protein and is included as a control, formulations 2 to 4 are examples of protein-containing methycellulose hyaluronic acid hydrogels of the present invention.

TABLE 3
sample formulation compositions

| | | | | Col- | Col- | |
|---|---|---|---|---|---|---|
| | | | | lagen | lagen | 1X |
| | Methyl- | Hyaluronic | Gelatin | I | II | PBS |
| Formulation | cellulose (g) | acid (g) | (mg) | (mg) | (mg) | (ml) |
| 1 | 32 | 2 | | | | 400 |
| (control) | | | | | | |
| 2 | 32 | 2 | | 100 | | 400 |
| 3 | 32 | 2 | 400 | | | 400 |
| 4 | 32 | 2 | 400 | | 10 | 400 |

### Example 5

Hydrogels with Chondrocytes

Samples of each of the four formulations of Example 4 are combined with equine chondrocytes to determine cell viability and chondrogenicity. Samples of equine chondrocytes are spun down to obtain pellets. Each pellet is resuspended in 100 μL DMEM/F12 cell culture medium in a 50 mL vial. To each vial is added an amount of the respective hydrogel solution at 4° C. to obtain a composition of 5 million cells/mL, and the vials are vortexed for 10 to 15 seconds to obtain a homogeneous mixture of cells in hydrogel. Each hydrogel/cell mixture is drawn into a syringe and transferred in aliquots of 200 to 300 μL into a 24-well transfer plate in which each well is fitted with a Transwell® insert (Corning #3472, 6.5 mm diameter, 3.0 μm pore size). To each well 400 to 500 μL of cell culture medium is added at the side of the well where the insert is located. The well plates are immediately incubated at 37° C. in a cell culture incubator, and the medium is replenished every 3 to 4 days.

DNA in each sample is measured at intervals of 1, 3, 7, 14, and 21 days using a Pico Green kit (Invitrogen catalog #P11496). The standard curve for the kit is shown in FIG. 2A, and the results are shown in FIG. 2B. The results illustrated in FIG. 2B indicate that the three samples that include the protein in the hydrogel in accordance with the invention exhibit better cell viability than the hydrogel formulation that do not include any protein.

### Example 6

### Evaluation of Mechanical Properties

Another set of samples of the four formulations of Example 4 are combined with equine chondrocytes in the manner described in Example 5, except that the cell pellets are resuspended in 10 mL fresh medium for cell counting using a hemacytometer. The hydrogel solutions are aliquotted to result in a concentration of 5 million cells/mL. Approximately 200 to 400 μL aliquots of combined hydrogel and cell solution are dispensed into the wells of 24-well plates, 12 aliquots for each hydrogel formulation. Aliquots are also made of samples without cells. To each well is added 500 μL of DMEM/F12 medium supplemented with 10% FBS+vitamin C and pen/strep. The well plates are immediately incubated at 37° C. in a cell culture incubator, and the medium is replenished every 3 days.

At intervals of 1, 7 and 14 days, samples without cells are removed from the medium, and the complex modulus is measured on a rheometer (MCR101, Anton Paar), using a frequency sweep analysis at about 10 Hz and 1% strain. The data are set forth in Table 4.

TABLE 4
complex modulus - no chondrocytes

| | Day 1 | | Day 7 | | Day 14 | |
|---|---|---|---|---|---|---|
| Formulation | Avg | Std dev. | Avg | Std dev | Avg | Std dev |
| 1 | 668.50 | 53.03 | 507.50 | 102.53 | 435.50 | 224.15 |
| 2 | 594.00 | 118.79 | 517.50 | 13.44 | 600.50 | 125.16 |
| 3 | 647.00 | 43.84 | 472.50 | 7.78 | 777.50 | 156.27 |
| 4 | 888.50 | 68.59 | 765.00 | 29.70 | 845.50 | 118.09 |

The same measurements are made on the hydrogel formulations with equine chondrocytes prepared in Example 6. The data are set forth in Table 5.

TABLE 5
complex modulus with chondrocytes

| | Day 1 | | Day 7 | | Day 14 | |
|---|---|---|---|---|---|---|
| Formulation | Avg | std | Avg | std | Avg | std |
| 1 | 452.50 | 14.85 | 377.50 | 30.41 | 601.50 | 111.02 |
| 2 | 374.00 | 7.07 | 343.50 | 16.26 | 385.50 | 38.89 |
| 3 | 443.50 | 7.78 | 463.50 | 20.51 | 472.50 | 36.06 |
| 4 | 552.00 | 91.92 | 447.50 | 58.69 | 718.00 | 28.28 |

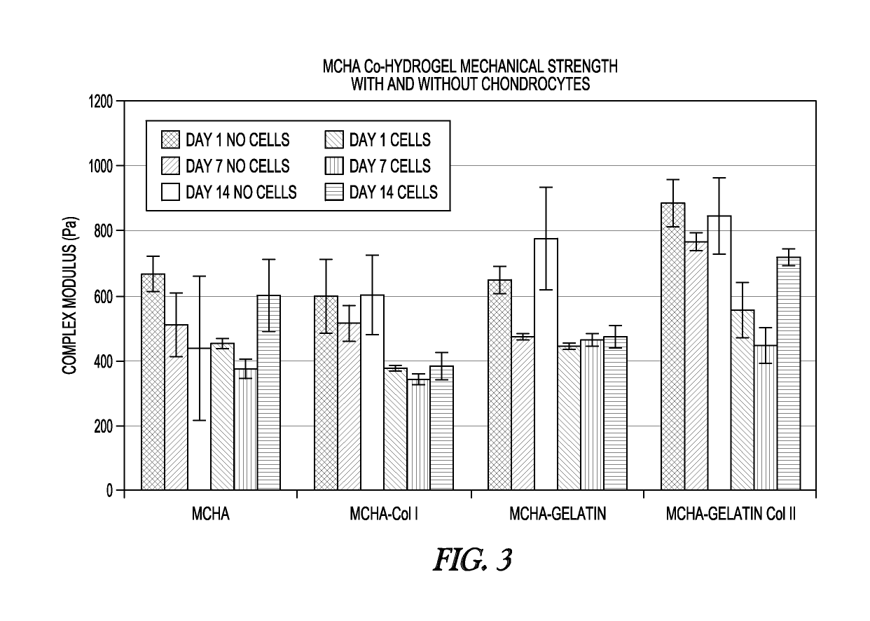

FIG. 3 is a graph summarizing the data of Tables 4 and 5.

### Example 7

### Evaluation of Hydrogel Degradation

The samples evaluated are those of Example 4, without added chondrocytes. Immediately after removal from storage at 4° C., samples of approximately 200 μL of hydrogel solution are aliquotted into eppendorf tubes containing 1 mL of DMEM/F12 medium supplemented with 10% FBS, vitamin C and pen/strep. The samples are incubated at 37° C. for a period of 1 to 21 days. In addition, two samples of each of the four formulations are evaluated two hours after aliquotting to obtain a baseline. For measurement, the medium is removed from the sample, and the sample is frozen at −80° C. before lyophilizing to determine an initial polymer dry weight. This process is repeated for the samples over a period of 21 days. Percent degradation is determined as % degradation(t)=(W_{d}(0)−W_{d}(t))/W_{d}(0)*100. The data so obtained are set forth in Table 6.

TABLE 6
hydrogel degradation

| | Form 1 | | Form 2 | | Form 3 | | Form 4 | |
|---|---|---|---|---|---|---|---|---|
| Day | % deg | stdev | % deg | stdev | % deg | stdev | % deg | stdev |
| 0 | 0.00 | 0.00 | 0.00 | 0.00 | 0.00 | 0.00 | 0.00 | 0.00 |
| 1 | 25.76 | 2.16 | 30.76 | 23.97 | 28.51 | 19.65 | 36.31 | 13.87 |
| 7 | 31.82 | 23.37 | 44.19 | 7.07 | 46.49 | 25.25 | 41.65 | 26.20 |
| 21 | 49.43 | 27.01 | 59.06 | 16.12 | 42.59 | 12.33 | 55.61 | 7.07 |

The data indicate that these hydrogel formulations of the present invention will degrade by approximately 50% to about 60% over a 21 day period.

There has thus been disclosed a hydrogel-forming composition suitable for administering therapeutic agents to areas of the body in need thereof. Methods of making and using the composition are also disclosed. Those skilled in the art will recognize other variations and equivalents of the composition and the components thereof, as well as the method of making and method of using, and all such variations and equivalents are intended to be encompassed within the scope of the claims appended hereto.

What is claimed is:

1. A hydrogel-forming composition, comprising: (a) a biocompatible thermosensitive polymer, (b) isolated hyaluronic acid, (c) an isolated extracellular matrix protein, and (d) an aqueous medium, said composition being substantially liquid at ambient temperature and gelable at temperatures above ambient temperature.

2. The composition according to claim 1, wherein the extracellular matrix protein is selected from any one or more of the group consisting of type I collagen, type II collagen, type A gelatin, type B gelatin, elastin, fibrillin, fibronectin, laminin, and proteoglycans.

3. The composition of claim 1 wherein said biocompatible thermosensitive polymer is selected from one or more of methyl cellulose; N-isopropylacrylamide (NiPAAM); poly(vinyl alcohol); poly(NiPAAM)/poly(ethylene glycol); poly(ethylene oxide-propylene oxide-ethylene oxide) (PEO-PPO-PEO); poly(ethylene glycol-lactic acid-ethylene glycol) (PEG-PLLA-PEG).

4. The composition of claim 1 wherein said biocompatible thermosensitive polymer comprises methyl cellulose.

5. The composition of claim 1 wherein said hyaluronic acid is present in an amount of about 0.2 to about 6 w/v % of said composition.

6. The composition of claim 1 wherein said extracellular matrix protein is present in an amount of about 0.001 to about 0.5 w/v % of said composition.

7. The composition of claim 1 wherein said biocompatible thermosensitive polymer is present in an amount of about 2 to about 12 w/v % of said composition.

8. The composition of claim 1 wherein said aqueous medium is phosphate buffered saline.

9. The composition of claim 8 wherein said aqueous medium is about 0.010 M phosphate buffered saline.

10. The composition of claim 1 comprising one or more therapeutic agents.

11. The composition of claim 10 wherein one of the therapeutic agents comprises mammalian cells.

12. A method of making a hydrogel-forming composition, comprising: (a) preparing a mixture of isolated hyaluronic acid and biocompatible thermosensitive polymer in an aqueous medium, and (b) adding an isolated extracellular matrix protein to the aqueous mixture.

13. The method of claim 12 wherein said biocompatible thermosensitive polymer is one or more of methylcellulose; N-isopropylacrylamide (NiPAAM); poly(vinyl alcohol); poly(NiPAAM)/poly(ethylene glycol); poly(ethylene oxide-propylene oxide-ethylene oxide) (PEO-PPO-PEO); or poly(ethylene glycol-lactic acid-ethylene glycol) (PEG-PLLA-PEG).

14. The method of claim 12 wherein said biocompatible thermosensitive polymer comprises methylcellulose.

15. The method of claim 12 wherein said hyaluronic acid is added in an amount of about 0.2 to about 6 w/v % of said composition.

16. The method of claim 12 wherein said extracellular matrix protein is added in an amount of about 0.001 to about 0.5 w/v % of said composition.

17. The method of claim 12 wherein said biocompatible thermo sensitive polymer is present in an amount of about 2 to about 12 w/v % of said composition.

18. A method of administering a therapeutic agent, comprising:
(a) preparing a hydrogel-forming composition comprising a biocompatible thermosensitive polymer, isolated hyaluronic acid, an isolated extracellular matrix protein, an aqueous medium, and a therapeutic agent, and
(b) administering said composition to a site in a mammal need of the therapeutic agent.

19. The method of claim 18 wherein said composition is administered as a liquid.

20. The method of claim 18 wherein said composition is administered as a hydrogel.
